SEQUENCE LISTING (3 sequences)

SEQ ID NO: 1 - protein, 363 aa, Homo sapiens
  MISC_FEATURE (1)..(363): gi 14043373
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 2 - DNA, 1559 bp, Homo sapiens
  misc_feature: gi 14043372
ggcacgaggcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaaaaaaaaaaaaaaaaaaaaaaa

SEQ ID NO: 3 - DNA, 1103 bp, Artificial Sequence
  Sequence variant of gi 14043372
ccatggcccagacgccggccttcgacaagccgaaagtagaactgcatgtccacctggacggttccatcaagccggaaaccatcctgtactatggccgtcgtcgcggtatcgccctgccggctaacacagcagagggtctgctgaacgtcattggcatggacaagccgctgaccctgccggacttcctggccaagtttgactactacatgcctgctatcgcgggctgccgtgaggctatcaaacgtatcgcctatgagtttgtagagatgaaggccaaagagggcgtggtgtatgtggaggtgcgctacagtccgcacctgctggccaactccaaagtggagccaatcccgtggaaccaggctgaaggggacctcaccccggacgaggtggtagccctcgtgggccagggcctgcaggagggtgagcgtgacttcggcgtcaaggcccgctccatcctgtgctgcatgcgccaccagccgaactggtccccgaaggtggtggagctgtgtaagaagtaccagcagcagaccgtggtggccattgacctggctggtgatgagaccatcccaggcagcagcctcttgccgggtcatgtccaggcctaccaggaggctgtgaagagcggcattcaccgtactgtccacgccggtgaggtgggctcggccgaagtagtaaaagaggctgtggacattctcaagacagagcgcctgggtcacggctaccacaccctggaagaccaggccctctataaccgtctgcgccaggaaaacatgcacttcgagatctgcccgtggtccagctacctcactggtgcctggaagccggacacggagcatgcagtcattcgcctcaaaaatgaccaggctaactactcgctcaacacagatgacccgctcatcttcaagtccaccctggacactgattaccagatgaccaaacgtgacatgggctttactgaagaggagtttaaacgtctgaacatcaatgcggccaaatctagtttcctcccagaagatgaaaagcgcgagctgctcgacctgctctataaagcctatggcatgccaccgtcagcctctgcaggtcagaacctctgataaggatcc

(19) United States
(12) Patent Application Publication
(10) Pub. No.: US 2004/0175804 A1
(43) Pub. Date: Sep. 9, 2004

(54) Process for preparing dideoxyinosine using adenosine deaminase enzyme

(76) Inventors: Paul M. Skonezny, Baldwinsville, NY (US); Michael Politino, Syracuse, NY (US); Suo W. Liu, Manlius, NY (US); Alfred W. Boyle, Manlius, NY (US); Jason G. Chen, Manlius, NY (US); Gregory L. Stein, Mallory, NY (US); Thomas Franceschini, Cicero, NY (US); Wendy L. Anderson, Mexico, NY (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/787,284

(22) Filed: Feb. 26, 2004

Related U.S. Application Data
(60) Provisional application No. 60/451,842, filed on Mar. 4, 2003.

(51) Int. Cl. C07H 19/167; C12P 19/30; A61K 31/7076

(52) U.S. Cl. 435/89; 536/27.8; 514/45

(57) ABSTRACT

A method of making didanosine (ddI) including the steps of: (a) obtaining an enzyme expressing ddA deaminase activity; (b) immobilizing the enzyme onto an insoluble support; (c) contacting the enzyme with a dideoxyadenosine (ddA) solution of at least about 4% weight volume ddA in water for a time and under conditions to produce a ddI solution; and (d) isolating the ddI from the ddI solution. Optionally, the ddI mother liquor is reused in subsequent runs to improve yield.

This application claims a benefit of priority from U.S. Provisional Application No. 60/451,842, the entire disclosure of which is herein incorporated by reference.

### FIELD OF THE INVENTION

The present invention relates generally to a method of making 2′,3′-dideoxyinosine (ddI) from 2′,3′-dideoxyadenosine (ddA), more particularly, the present invention relates to a method of making ddI using adenosine deaminase enzyme (ADA) derived from a human ADA nucleotide sequence.

### DESCRIPTION OF THE BACKGROUND ART

Dideoxynucleosides are relatively stable nucleoside analogs. The dideoxynucleoside 2′,3′-dideoxyinosine (ddI) has been shown to have useful pharmacological activity as antiviral agents. Hartman, et al., Clin. Pharmacol. Ther. 47:647 (1990). In particular, ddI has been shown as useful when used alone or in combination with 3′-azido-2′,3′-dideoxythymidine (AZT) in the treatment of AIDS. Use of ddI has become increasingly important in light of the development of AZT resistant strains of human immunodeficiency virus.

Efforts to synthesize dideoxynucleosides in the laboratory have been reported, such as the deoxygenation of nucleosides at the 2′ or 3′ position. Chem. Pharm. Bull., 22:128 (1974). However, chemical synthesis is difficult due to steric hinderance and the instability of nucleosides under temperature and pH conditions normally required to drive reactions. Furthermore, the starting materials for the synthesis are costly and not available in bulk. To date, it has not been possible to scale up laboratory scale synthesis of dideoxynucleosides to industrial scale commercial production of these compounds.

A number of processes are known for industrial scale production of ddI by enzymatic deamination of dideoxyadenosine (ddA) by adenosine deaminase. Synthesis of ddI by deaminating 2′,3′-dideoxyadenosine using bovine adenosine deaminase has been reported. Webb et al., Nucleosides & Nucleotides, 7(2): 147-153 (1988). Scale up of a process of synthesizing ddI using bovine adenosine deaminase has also been reported, including optimization of parameters, such as solvents and buffers. Nucleosides & Nucleotides, 10(7): 1499-1505 (1991).

U.S. Pat. No. 5,011,774 to Farina et al., discloses a process in which an anomeric mixture of (D)-2′,3′-dideoxyadenosine is reacted with an ADA in a suitable solvent to selectively favor the rate of enzymatic deamination of the more active β-anomer of ddI. The processes uses commercially available ADA derived from calf spleen. This process avoids the steps of chromatographic and crystallization techniques necessary to separate out the undesirable α-anomer. However, a possible disadvantage of using this process is the risk of transmission of Transmissible Spongiform Encephalopathy (TSE) when bovine sources of the enzyme are used.

U.S. Pat. No. 4,970,148 to Yokozeki et al. discloses a process in which 2′,3′-dideoxyadenosine (ddA) is contacted with a culture solution of a microorganism containing ADA enzymes capable of converting the ddA to ddI. The process uses a culture solution of whole microorganisms, cell homogenates, or products of cells treated with lysozyme, salt, surface active agents, or the like as the source of the ADA. One disadvantage of this method is that the natural source of the enzyme is inherently unstable at a pH in excess of 8 and requires strict pH control. When performed at a pH less than 8, the product ddI is only soluble in very dilute concentrations (<1% weight volume). As a result, the method produces very little ddI per batch. Furthermore, the ddI so derived must undergo extensive purification procedures to remove residual protein contamination in order to obtain the purified ddI product. These purification procedures are costly and can result in loss of product and reduced yields.

Japanese Patent Applications No. 5-219978 to Noguchi et al., discloses a method of producing nucleic acid related substances such as ddI including cloning the gene coding for the appropriate enzyme, constructing an expression vector with regulatory sequences resulting in high expression of the gene, transforming a microorganism with the expression vector to form a transformant, inducing expression of the cloned gene, and extracting and isolating the enzyme so expressed. The isolated enzyme is then used to produce the desired nucleic-acid-related substance by reaction with a suitable starting material, such as ddA. This method produces a 100 fold increase in the amount of enzyme available for use as compared to the Yokozeki patent. However, the enzymes isolated according to this method are microbial enzymes. As such, at least with respect to the ADA, the enzyme lacks stability at pH values greater than 8. As a result, in order to obtain acceptable yields, the pH must be closely regulated. Furthermore, this method produces a product that is in intimate contact with the ADA. The reaction mixture is contaminated with impurities such as unreacted ddA, nucleic acid by-products as well as the ADA and the product. Consequently, extensive purification methods must be performed in order to isolate the ddI from the reaction mixture. Such purification methods typically require repeated liquid chromatography or thin layer chromatography. These purification procedures are not amenable to commercial scale up.

U.S. Pat. No. 4,962,193 to Yokozeki et al., discloses a method of purifying ddI from a process using an enzyme which uses a porous, non-polar resin to adsorb the ddI onto the resin, separating the resin from solution, and fractionally eluting the adsorbed ddI to obtain purified product. However, this method is preceded by treatment to remove proteins and concentrate and filter the solution containing the product prior to the final purification step. As a result, this method can be costly and time consuming.

There is a present need for an industrial scale method of making ddI that provides satisfactory yield without the risk of transmission of TSE or the need to undergo extensive purification procedures to remove residual protein, and that uses an enzyme which is stable over a broad pH range.

### SUMMARY OF THE INVENTION

The present invention is generally directed to a method of making ddI by contacting ddA with an enzyme having ddA deaminase activity, which is immobilized on an insoluble support. The present invention provides a method of making didanosine (ddI) including the steps of: (a) obtaining an enzyme expressing ddA deaminase activity; (b) immobilizing the enzyme onto an insoluble support; (c) contacting the enzyme with a dideoxyadenosine (ddA) solution of at least about 4% weight volume ddA in water for a time and under conditions to produce a ddI solution; and (d) isolating the ddI from the ddI solution. Optionally, the resulting ddI mother liquor is reused in subsequent runs to improve yield.

### DETAILED DESCRIPTION OF THE INVENTION

The present invention is directed to methods of making ddI from ddA in a cost effective and reliable manner which avoids the shortcomings of the prior art methods.

Previous methods of preparing ddI from ddA involve using commercially available ADA, such as bovine ADA (available from Sigma) or ADA derived from growing E. coli transformed with microbial ADA. The methods involve admixture of the enzyme in solution with ddA to convert the ddA to ddI. Next, the ddI must be removed from a solution containing a variety of contaminants, including the enzyme. This requires substantial purification and separation to obtain ddI from the contaminated reaction mixture.

In contrast to these prior art methods, the present invention uses ADA, or other enzyme capable of deaminating ddA, in an immobilized state. The immobilization assists in improving stability of the enzyme in the reaction mixture. More importantly, the immobilization allows for easy separation of the ddI final product from the reaction mixture because a main contaminant, namely the enzyme, remains immobilized on an insoluble support.

The invention further provides a new source of ADA. In a preferred embodiment of the invention, ddI is made from ddA using ADA derived from growing organisms transformed with human ADA or conservative derivatives thereof. One advantage of the present method is that human ADA is more stable than microbial ADA over a broader pH range. When the reaction can be performed at a pH in excess of about 8 the ddA remains stable and conversion efficiency to ddI is improved. An additional advantage is that the ddI remains in solution at these pH's much more readily than at a pH of less than about 8. The characteristic of improved solubility of the product ddI at high pH (>8) helps to enable reaction at higher concentrations, and thus provide improved yields of ddI.

The inventive methods use human ADA enzyme or other enzyme having ddA deaminase capability, that has been immobilized onto an insoluble support. The present invention takes useful advantage of the improved stability afforded the enzyme which results from such immobilization. As a result, the reaction of ddA to ddI can proceed at pH ranges that typically denature or otherwise interfere with the activity of the enzyme. Additionally, immobilization of the ADA imparts a convenient characteristic to the ADA, namely the ability to separate the enzyme from the reaction product by simple filtration methods.

Preparing Adenosine Deaminase (ADA)

The ADA of particular interest in the present invention is human ADA or a conservative variant thereof, having amino acid sequence SEQ ID NO: 1 (Genbank Accession number gil14043373). The human version of ADA was selected due to its superior structural stability to that of microbial origin. In particular, the human ADA maintains significant activity in a relatively wide pH and range as compared to microbial ADA. Furthermore, the human ADA is more resistant to degradation at elevated temperatures as compared to that of microbial origin. The DNA sequence of the human ADA is published as a cDNA sequence derived from human mRNA which is a 1,533 base sequence. See, Gwendolyn, S. et al., Mol. and Cellular Biology, 4(9):1712-1717 (1984).

The published sequence, or certain conservative variations thereof, may be used to code for and obtain the desired ADA. SEQ ID NO:2 (Genbank Accession number gil14043372) is the published human cDNA sequence. Desirably, especially when using E. coli as the host, SEQ ID NO:3 (Genbank Accession number gil140433732) is used. SEQ ID NO:3 is a conservative variant of the published human cDNA in which codon preference substitutions have been made for arginine, glycine, leucine, isoleucine and proline. As the genetic code is degenerated, these codon substitutions do not result in an alteration of the amino acids coded for by the sequence. Rather, the substitution improves recognition of the codons by E. coli. The following codon preference substitutions may be used:

TABLE 1
Codon Preference Substitutions

| | | |
|---|---|---|
| Amino Acid | Mammalian Codon | Substitute Codon |

| | | |
|---|---|---|
| Arginine | AGG | CGT |
| ″ | AGG | CGC |
| ″ | CGG | CGC |
| ″ | CGG | CGT |
| ″ | AGA | CGC |
| Glycine | GGA | GGT |
| ″ | GGG | GGT |
| ″ | GGG | GGC |
| Isoleucine | ATA | ATT |
| Leucine | TTA | CTG |
| ″ | CTA | CTC |
| ″ | CTC | CTG |
| ″ | CTT | CTG |
| Proline | CCC | CCG |
| ″ | CCA | CCG |
| ″ | CCT | CCG |
| ″ | CCA | CCG |

The invention further includes use of sequences including other minor modifications, and all naturally occurring alleles, of the amino acid sequence set forth in SEQ ID NO: 1 that result in enzymes which have substantially equivalent activity. Modifications may be deliberate, as by site-directed mutagenesis, or may be spontaneous mutations. Alleles may be from any species. Preferred alleles are of human origin. The invention includes use of all of these polypeptides so long as the activity of the enzyme in deaminating ddA is retained.

For example, the invention also includes conservative variations or equivalent variants of SEQ ID NO: 1. The terms “conservative variation” and “equivalent variant” as used herein denote the replacement of amino acids by other amino acids that have similar chemical and biological properties, or that are generally considered equivalent.

For example, it is known in the art to substitute amino acids in a sequence with equivalent amino acids, i.e. conservative variations. Groups of amino acids normally considered to be equivalent are:

Ala (A), Ser (S), Thr (T), Pro (P), Gly (G);

Asn (N), Asp (D), Glu (E), Gln (Q);

His (H), Arg (R), Lys (K);

Met (M), Leu (L), Ile (I), Val (V); and

Phe (F), Tyr (Y), Trp (W).

Substitutions, additions, and/or deletions in the enzyme sequences may be made as long as the function of the ADA used in the methods of the invention is maintained. Equivalent enzymes will normally have substantially the same amino acid sequence as the native enzyme. An amino acid sequence that is substantially the same as another sequence, but that differs from the other sequence by means of one or more substitutions, additions and/or deletions, is considered to be an equivalent sequence, equivalent variant or conservative variation. Preferably, less than 25%, more preferably less than 10%, of the number of amino acid residues in a sequence are substituted for, added to, or deleted from the proteins of the invention.

The human ADA can be prepared by methods known in the art. Such methods include biological synthesis and chemical synthesis. In biological synthesis, the enzyme may be isolated directly from cells. Alternatively, it is known to prepare the enzyme by providing DNA that encodes the enzyme, amplifying or cloning the DNA, expressing the DNA in a suitable host, and harvesting the enzyme. For example, the enzyme may be translated either directly or indirectly from a cDNA encoding the enzyme amino acid sequence. In chemical synthesis, the four bases are used as raw materials to assemble the known amino acid sequence.

A. Obtaining the Nucleic Acid Sequence which Codes for ADA

The DNA encoding the ADA may be derived from an appropriate cDNA library by methods known in the art. See, for example, Gwendolyn, S. et al., Molecular and Cellular Biology, 4(9):1712-1717 (1984), the entirety of which is herein incorporated by reference. The sequence has been assigned GenBank Accession No. GI: 14043372.

Generally, the entire DNA strand or additional fragments of the DNA can be isolated by using a known DNA or a fragment thereof as a probe. To do so, restriction fragments from a genomic or cDNA library may be identified by Southern hybridization using labeled oligonucleotide probes. For example, DNA encoding the enzyme can be isolated from human homogenated tissue by using a fragment of the known sequence to prepare one or more oligonucleotide probes. The probe is labeled and used to screen a genomic or cDNA library in a suitable vector, such as phage lambda. The cDNA library may be prepared from mRNA by known methods, such as those described in Gubler and Hoffman, Gene, 25:263-270 (1983). Oligonucleotide probes can be used to screen cDNA libraries from different tissues. The oligonucletide probe should be labeled so that it can be detected upon hybridization to DNA in the library being screened. These methods are well known in the art. The DNA isolated is sequenced, and the sequence used to prepare additional oligonucleotide probes. This procedure may be repeated to obtain overlapping fragments until a complete open reading frame is produced.

Methods for DNA amplification and cloning are well known in the art. See, for example, Sambrook and Russel, Molecular Cloning A Laboratory Manual, 3^{rd} Ed., Cold Spring Harbor Laboratory, New York (2001), the entirety of which is herein incorporated by reference. It is convenient to amplify the clones in the lambda-gt10 or lambda-gt11 vectors using lambda-gt10 or lambda-gt11-specific oligomers as the amplimers (available from Clontech, Palo Alto, Calif.). Other amplification procedures that are well known in the art such as ligase chain reaction (LCR), Repair Chain Reaction (RCR), and PCR oligonucleotide ligation assay (PCR-OLA) can also be used to amplify the nucleic acids of the invention.

As an alternative to the aforementioned biological synthesis of the gene, it is possible to chemically synthesize the gene, based on the reported DNA sequence using methods known in the art. Such methods include those described in “Modern machine-aided methods of oligonucleotide synthesis”, Brown T and Dorcas, J. S., In Oligonucleotide and Analogues a Practical Approach, Ed. F. Eckstern, IRL Press, Oxford UK (1995), the entirety of which is herein incorporated by reference. DNA can also be synthesized by preparing overlapping double-stranded oligonucleotides, filling in the gaps, and ligating the ends together. See, generally, Sambrook et al., and Glover, D. M. and Hames, B. D., eds. Cloning, 2nd Ed., Vols. 1-4, IRL Press, Oxford, UK (1995).

In the event there is any concern regarding potential viral contamination related to human sources of DNA, such as for compliance with regulatory requirements, then the chemical synthesis method of obtaining the DNA is preferred.

B. Expressing the ADA

The recombinant DNA molecules, obtained as described above, contain polynucleotide sequences encoding human ADA. This recombinant DNA may be cloned in a suitable host cell and expressed by methods well known in the art. Generally, the cloned gene is provided with expression vectors which direct expression of the enzyme in an appropriate host cell. The enzyme may be then be recovered from the host cell. See, Sambrook et al. (2001), for methods relating to the manufacture and manipulation of nucleic acids.

The amplified or cloned DNA can be expressed in a suitable vector, preferably an expression vector, by methods known in the art. See, generally, Sambrook et al. (2001). Expression vectors are capable of directing the expression of genes to which they are operably linked. Expression vectors of utility in recombinant DNA techniques are often in the form of plasmids. However, the invention may include other forms of expression vectors, such as viral vectors (e.g., replication defective retroviruses, adenoviruses, and the like), which serve equivalent functions. Preferably, the expression vector is a plasmid, as disclosed in U.S. Pat. No. 6,068,991, the entirety of which is herein incorporated by reference.

Vector DNA, preferably in the form of expression vectors including regulatory sequences, can be introduced into prokaryotic or eukaryotic host cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, liposome mediated transfection (lipofection), or electroporation. Suitable methods for transforming or transfecting host cells can be found in Sambrook, et al. (supra), and other laboratory manuals.

The expression vectors preferably contain at least one expression control sequence that is operatively linked to the DNA sequence or fragment to be expressed. The control sequence is selected on the basis of the host cells to be used for expression and inserted in the vector in order to control and to regulate the expression of the cloned DNA sequence.

Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology, 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences).

Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, (e.g., Pho5), the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Examples of suitable inducible non-fusion E. coli expression vectors include regulatory sequences such as pTrc (Amann et al., (1988) Gene 69:301-315) and pET 11d (Studier et al., Gene Expression Technology: Methods to Enzymology 185, Academic Press, San Diego, Calif. (1990)). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS 174(DE3) from a resident lambda prophage harboring a T7 gn1 gene under the transcriptional control of the lacUV 5 promoter.

Suitable host cells can be any prokaryotic (e.g., E. coli) or eukaryotic cell (e.g., insect cells, yeast or mammalian cells). Some suitable prokaryotic hosts include, for example, E. coli, such as E. coli SG-936, E. coli HB 101, E. coli DH5α, E. coli X2282, E. coli DHI, and E. coli MRC1, E. coli BL21, Pseudomonas sp., Bacillus sp., such as B. subtilis, and Streptomyces sp. Suitable eukaryotic cells include yeasts and other fungi, insect, animal cells, such as COS cells and CHO cells, human cells and plant cells in tissue culture. Preferably, the host cell is E. coli.

It will be appreciated by those skilled in the art that the design of the expression vector can depend on such factors as the choice of the host cell to be transformed, the level of expression of protein desired, and the like. The expression vectors of the invention can be introduced into host cells to thereby produce ADA, including fusion proteins or peptides, encoded by nucleic acids as described herein.

Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL, (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) pp. 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques or site-specific mutagenesis.

The cells are grown under conditions well known in the art. Expression of the cloned gene is induced to express large amounts of the enzyme. Preferably, an E. coli expression vector is used including the lac system promoter system. Expression of ADA is preferably induced using IPTG.

C. Isolation and Purification of ADA

Generally, enzymes can be isolated from a solubilized cell fraction by standard methods. Some suitable methods include precipitation and liquid chromatographic protocols such as ion exchange, hydrophobic interaction, and gel filtration. See, for example, Methods Enzymol.—Guide to Protein Chemistry, Deutscher, Ed., Section VII pp. 182-309 (1990); and Scopes, Protein Purification, Springer-Verlag, New York (1987), which are herein incorporated by reference.

Alternatively, purified material is obtained by separating the enzyme on preparative SDS-PAGE gels, slicing out the band of interest and electroeluting the protein from the polyacrylamide matrix by methods known in the art. The detergent SDS is removed from the protein by known methods, such as by dialysis or the use of a suitable column, such as the Extracti-Gel column from Pierce. Mixtures of enzymes can be separated by, for example, SDS-PAGE in accordance with the method of Laemmli, Nature 227:680-685 (1970). Such methods are well known in the art.

There are no particular limitations to the method of purifying the protein so long as undesirable contamination is effectively removed. An economical method is to break down the cells by passing the fermentation broth through a microfluidizer to release the enzyme from the cells. Addition of a filter aid and a flocking agent (e.g., PEI, available from VWR International, South Plainfield, N.J.) to the stirred broth renders the contaminants such as proteins and other cellular debris insoluble. A suitable filter aid is CELITE (available from World Minerals, Inc., Santa Barbara, Calif.). The soluble enzyme may then be removed from the broth by filtration with an appropriately sized filter. Desirably, the filter will allow the soluble active enzyme to pass through while allowing insoluble fractions of cellular proteins and other contaminants to be retained by the filter. The enzyme can then be concentrated in solution using an ultrafilter. A 30,000 molecular weight cut off (MWCO) filter is useful for this purpose.

Assay ADA Activity

The enzyme so derived should be assayed for activity. As used herein, a unit (U) of enzymatic activity is that amount of enzyme which will deaminate 1 μmol of ddA per minute at 37° C. Desirably, the final enzyme titer is from about 650-750 U/ml. The assay may be performed by adding enzyme to a 2.4% solution of ddA at 37° C. The reaction is allowed to proceed for 15 min while gently stirring. Tetrahydrofuran is added to stop the reaction. A sample is taken and run on HPLC to determine the amount of ddI formed.

Immobilizing ADA

Once the enzyme is purified, it is desirable to treat the enzyme solution with an appropriate buffer to a pH of about 7.3 to about 7.6. There are no particular limitations to the type of buffer used, although phosphate buffer is preferred. Desirably, the solution is diluted with buffer to an activity of from about 250 to about 350 U/ml.

The purified enzyme is first immobilized onto a support that is insoluble in the reaction solution before reaction with ddA. There are no particular limitations as to the type of support used so long as the enzyme may be immobilized thereon. In general, the aforementioned dilute solution of buffered enzyme is added to a solution containing an insoluble support, some of which require activation by an activating agent such as a crosslinking reagent. The crosslinking reagent serves to covalently bond the ADA to the support via an amine group on the ADA.

The support remains insoluble, and maintains the enzyme insoluble in the reaction solution, during the course of the reaction. Desirably, the support is a solid resin material having a diameter of about 250-600 microns. Suitable supports include, for example, IPS-400 (available from U.O.P., Des Plaines, Iowa) or EUPERGIT (available from Rohm America Inc, Piscataway, N.J.).

There are no particular limitations to the crosslinking agent as long as it covalently attaches the enzyme to the support. Selection of crosslinking agents will depend on the support selected and will be readily apparent to those having skill in the art. Suitable combinations of resin support and crosslinking agents are Celite and glutaraldehyde, IPS-400 and glutaraldehyde, Sepharose and CNBr and resin supports functionalized with either primary amines or carboxyls and carbodiimides. Other appropriate combinations of solid and crosslinking reagents will be apparent to those having ordinary skill in the art.

The immobilization of enzyme onto the support may be performed in a batch or continuous process. A batch process may be performed, for example, by mixing the buffered enzyme solution with activated support for a number of hours. The immobilized enzyme is then collected using a simple filtration technique. The particle retention size will be determined by the size of the solid support. For use with IPS-400, a filter having a particle size retention of from about 20 μm to about 30 μm is useful. Vacuum may be applied to speed recovery, however the support should not be dried.

Alternatively, the activated support can be collected on a filter with particle retention size sufficient to retain the solid support. The buffered enzyme solution is then passed over the support. The enzyme mother liquor from the filtration will be passed through the filter repeatedly to maximize immobilization of the enzyme. In a continuous process, the solid support may be slurried and poured into a chromatography column. After recovery the immobilized enzyme is rinsed with water to remove any impurities or unbound enzyme. Preferably, a titer of the immobilized enzyme is at least about 40U.

Reacting ddA with ADA

The immobilized ADA is then admixed with a ddA solution to obtain ddI. During reaction of ddA to ddI, ammonia is generated. Since ddA is added to the enzyme in more concentrated solutions than in the prior art, the ammonia byproduct in the mixture can cause the pH to be elevated in a range of from about 9.2 to about 9.5. Use of microbial ADA or unbound ADA under these conditions is not expected to proceed to completion because the enzyme will become inactivated at these elevated pH's. However, use of the human version of the ADA immobilized on a solid support diminishes such degradation. As a result, the ADA retains activity and the reaction proceeds at a faster, more productive pace, than has heretofore been possible.

The reaction is performed at a temperature of from about 20° C. to about 50° C. Desirably, the reaction is maintained at a temperature of about 25-30° C. The reaction can be performed at lower temperatures, however this will result in a longer reaction time. Performing the reaction at temperatures in excess of about 50° C. can result in impairment of enzymatic activity and/or denaturing of the enzyme.

Commercially available ddA (available from Ajinomoto, Tokyo, Japan) is added to the immobilized ADA in water in a batch or continuous bulk process. The reaction may proceed using concentrations of ddA well above those used in methods in which the ADA is not immobilized. An acceptable range of concentration of ddA in the reaction solution is from about 1% to about 15%. Desirably, a solution of about 4% to 10% of ddA, more desirably a solution of about 5-6% ddA in water is added to the immobilized ADA. The reaction is allowed to proceed under conditions and for a time so that until about 1% or less of the ddA remains.

In a batch process, the ddA will be added to a suspension of immobilized ADA in water and allowed to react. Complete reaction should take about 5 to 8 hours. Once completed, the immobilized ADA can be recovered for reuse by filtration followed by washing with water. Furthermore, the mother liquor, after removal of ddI product, can be reused to maximize yield.

In a continuous process, a ddA solution may be added to a column packed with immobilized ADA. Desirably, the height to diameter ratio will be about 6, although the ratio is not critical. The ddA solution will be added at a rate and for a time so that about 1% or less of the ddA remains. Complete reaction should take about 120 hours. The flow rate will be dependent on the size of the column, concentration of ddA, and temperature of the reaction. Alternatively, a continuous process may be used in which the ddA solution is recycled through the packed column. The ddA solution will be recycled at a rate and for a time so that about 1% or less of the ddA remains. The flow rate will be dependent on the size of the column, concentration of ddA, and temperature of the reaction. Selection of the rate of introduction of ddA solution will be readily apparent to those of skill in the art.

Recovering ddI

After reacting the ddA to ddI, the ddI is present in solution in the form of an ammonium salt at high pH (>8). In the recovery step, the ddI is removed from solution. This is achieved by crystallizing the ddI out of solution. A simple distillation process may be used to drive off ammonia, a side product of reaction and produce the free acid form of ddI. The distillation can be performed sequentially with a first distillation bringing the solution to a concentration of about 10-12% (based on initial ddA) followed by addition of water and further distillation until the concentration again reaches about 10-12% and the pH of the ddI slurry is less than about 8. The suspension may then be cooled to about 0-5° C. and held for at least one hour.

The ddI can be filtered and the cake washed with acetone. The solids may then be dried to a constant weight, for example under vacuum at about 45-50° C. Desirably, the reaction mother liquor may be retained for reuse in the batch or continuous processes reacting ddA to ddI. Additionally, the aqueous wash may also be reused.

Yields of about 82% may be obtained without any recycling. However, when the reaction mother liquor is recycled, yields can be increased to about 96-99%. The resulting ddI is greater than 99% pure.

The following Examples are intended to show the practice of the invention and are not intended to restrict the scope of the present invention. All percentages are in weight/volume unless otherwise indicated.

The entire disclosure of each document cited (including patents, patent applications, journal articles, abstracts, laboratory manuals, books, Genbank Accession numbers, SWISS-PROT Accession numbers, or other disclosures) in the Background of the Invention, Detailed Description, Brief Description of the Figures, and Examples is hereby incorporated herein by reference in its entirety. Further, the hard copy of the Sequence Listing submitted herewith, in addition to its corresponding Computer Readable Form, are incorporated herein by reference in their entireties.

### EXAMPLE 1

### Preparation of Recombinant E. coli Transformed with a Human Adenosine Deaminase (ADA) Gene

E. coli expression plasmid, pBMS2000, was digested with restriction enzymes, BspHI and BamHI, and fractionated on 0.7% agarose gel. The fragment corresponding to the 4.5 Kb was excised from the gel, eluted, concentrated by ethanol precipitation. The synthetic human ADA DNA was excised from the plasmid containing the gene coding for the human ADA gene with the restriction enzymes, Nco I and BamHI. The NcoI-Bam HI fragment containing the synthetic human ADA gene was ligated to the 4.5 Kb fragment of pBMS2000 obtained by digestion with the restriction enzymes, BspHI and BamHI. The ligated DNA was transformed into E. coli host, BL21. The transformed cells were plated onto Lauria Broth agar plates supplemented with 30 μg of neomycin sulfate. Restriction enzyme analysis was performed on some of the colonies as well as SDS-PAGE analysis. One of the colonies with the correct restriction analysis and the enzymatic activity was selected to be carried forth.

### EXAMPLE 2

### Fermentation of Recombinant E. coli Containing the Synthetic Human Adenosine Deaminase Gene

Fermentation Medium Formulations

| | |
|---|---|
| Reagent | Amount |

Seed medium (per 100 ml)

| | | |
|---|---|---|
| Soytone | 1 | g |
| Yeast extract | 0.5 | g |
| NaCl | 0.16 | g |
| Neomycin sulfate (657 mg/g) | 2.0 | mg |

Base medium (per liter)

| | | |
|---|---|---|
| K₂HPO₄ | 14 | g |
| Citric acid | 2 | g |
| Yeast extract | 3.2 | g |
| NaCl | 1.6 | g |
| MgSO₄—7H₂O | 2.2 | g |
| Neomycin Sulfate | 18 | mg |
| Glycerol | 3.4 | ml |
| PPG | 0.2 | ml |

Feed medium (per liter)

| | | |
|---|---|---|
| Cerelose | 200 | g |
| Yeast extract | 100 | g |
| Neomycin sulfate | 18 | mg |

PH Control - Base

| | | |
|---|---|---|
| Concentrated NH₄OH | 250 | ml |
| H₂O | 750 | ml |

PH Control - Acid

| | | |
|---|---|---|
| 85% Phosphoric acid | 200 | ml |
| Water | 800 | ml |

A. Preparation of seed stock

0.2 ml of thawed recombinant E. coli is inoculated into a 500 ml Erlenmeyer flask containing 100 ml of the seed medium as described above. This is incubated by shaking the flask in a gyratory incubator at 300 rpm at 28° C. for 24 hours to prepare an inoculum.

B. Fermentation

50 ml of the inoculum is inoculated into a 5 liter fermenter having a 1 liter working volume. Operating conditions are: a temperature of 28° C.; 1000 rpm agitation; aeration of 1 vvm; dissolved oxygen (DO) setting is recorded and defined at 100%; pH controlled with NH₄OH and phosphoric acid to pH 6.8 to 7.2.

2 liters of the feed medium is prepared as follows. Ramp feeding is initiated after 6 hours using a feeding rate of 5 ml/hr increasing at 1.2 ml/hr/hr. The cells are allowed to grow for 24 hours with monitoring. Air flow, feed rate, and temperature are adjusted as required to control DO to greater than or equal to 20% of the initial setting. The temperature of the culture is then decreased to 16° C. The cells are induced with isopropyl β-D-thiogalactoside (IPTG) to the final concentration of 80 μg/ml between 16-18 hours post-inoculation. The optical density at 600 nm is between 20-30. Monitoring and feeding is continued for 40-48 total hours or until the feed is utilized. The broth contains 84 units/ml of solution. (1 unit (U)=1 μmol of ddA deaminated/min at 37° C.).

### EXAMPLE 3

### Isolation and Immobilization of the Human ADA from the Recombinant E. coli Fermentation

10 L of fermentation broth is passed through a microfluidizer (M−1 Y model, available from Microfluidics, Newton, Mass.). Operating pressure is at 12,000-20,000 psi until at least 90% of the activity is released from the cell. Activity is measured by taking a portion of the microfluidized broth, centrifuging the sample, and measuring the activity in the supernatant portion. The amount of activity in the supernatant represents the amount of activity released. Normally one pass through the microfluidizer is required.

To 10 L of well-stirred microfluidized broth, is added 1.5 kg of CELITE (final concentration 15% w/v). Then, 0.30 L of 10% aqueous polyethyleneimine (PEI) (final concentration 0.3% v/v) is added. This is stirred for at least 30 minutes then filter to remove the insolubles. The cake is washed with 3.0-4.0 L of water.

The clarified filtrate is ultrafiltrated through a 30,000 MWCO filter cassette (Pellicon 2 unit, polyethersulfone low protein binding cassette, 0.5 m² filter area, available from Millipore, Bedford, Mass.) to a final enzyme titer of between 650-750 U/ml. The sample is diluted with 1.25 L of 50 mM phosphate buffer pH 7.3-7.6 (to an enzyme activity of 250-350 U/ml).

To 550 g IPS-400 protein immobilization support, is added 2.75 L of 2.5% glutaraldehyde. This is gently stirred at room temperature for 2 hours and decanted or filtered onto a size 60 mesh filter. The activated support is washed 10 times with 4 L water.

To the diluted enzyme solution (˜0.95 L) is added 1.10 kg activated IPS-400 immobilization support and gently stirred for 2 hours at 20-25° C. The immobilized enzyme is collected on a filter having an effective pore size sufficient to retain the immobilization support and washed with 5 volumes of water.

Alternatively, the IPS-400 is slurried in tap water and collected on a Buchner funnel fitted with fast flow filter paper with 20 to 30 μm particle size retention (grade 604 or 415). Vacuum is applied to remove excess water but the support is not dried. The diluted enzyme is added to the resin, vacuum (20″ Hg) is applied and the mother liquor collected. Vacuum is stopped, the mother liquor sampled (pass #1) for activity then added back to the support and vacuum is reapplied. This is repeated four more times for a total of five passes over the IPS-400. After the 5^{th} pass, 1.5 L of water is added to the resin and vacuum applied to wash the immobilized enzyme.

As another alternative, the IPS-400 is slurried in tap water and poured into a chromatography column. The bed is allowed to settle, then water is passed through the column (up flow) to rinse the support. The diluted enzyme solution is pumped through the column (1 to 25 mil/min.). After addition of the enzyme, 1.5 L of water is pumped over the column to wash the immobilized enzyme.

The immobilized enzyme assays show an enzymatic activity of ˜100 U/g. (1 unit=1 μmol of ddA deaminated/min at 37° C.).

### EXAMPLE 4

### Preparation of ddI from ddA Using the Recombinant Human ADA Immobilized Enzyme (Batch)

100.0 g ddA is added to 1900 ml of water at 30° C. (mother liquor from previous runs can also be used). After the ddA has been added (complete dissolution is not necessary), 4000 U of immobilized rec human adenosine deaminase is added. The reaction is maintained at 30° C. while stirring. The reaction is complete (approximately 5-8 hours) when the level of residual ddA is less than or equal to 1% of the original amount of ddA added. The enzyme solids are removed by filtration (20-30 μm media) and the enzyme cake is washed with 100 ml water. The used enzyme is held as a wet cake at 0-5° C. (for up to 72 hours) and can be recycled to another batch. The ddI solution is filtered through a CUNO filtration pad (0.4 to 1 micron), then through 0.2 micron filter media.

### EXAMPLE 5

### Preparation of ddI from ddA Using the Recombinant Human ADA Immobilized Enzyme (Continuous Column)

A column (h/d ratio=6) is slurry packed with 380 U of immobilized rec human adenosine deaminase with water. The column is washed with 30 mM of NH₄OH to pH 9.5 then with water to pH 6.5-7.5. While maintaining the temperature at 20° C., a 4% solution of ddA in water is passed through the column at a rate of 7.2 mil/min. for 120 hours. The effluent from the column is at pH 9.2-9.5 and contains >99% ddI with <1% ddA remaining. The resulting ddI solution is filtered through a CUNO pad and a 0.21μ filter before final isolation.

### EXAMPLE 6

### Preparation of ddI from ddA Using the Recombinant Human ADA Immobilized Enzyme (Recycle Column)

A column (h/d ratio=2.7) is slurry packed with 1000 U of immobilized rec human adenosine deaminase with water. 25.0 g of ddA is dissolved in 475 ml of water (or a mother liquor from previous batch, diluted with water to 475 ml) at 30° C. in a 3-neck round bottom flask equipped with a mechanical stirrer. The ddA solution is circulated at 30° C. through the enzyme column at −50 mL/min (circulation speed can be varied as necessary).

The reaction is complete (approximately 3-9.5 hours) when the level of residual ddA is less than or equal to 1% of the original amount of ddA added by HPLC analysis. The column is then rinsed with 25 ml of water and can be held at 0-5° C. (for up to 72 hours) for reuse. The resulting ddI solution is filtered through a CUNO pad and a 0.2 μm filter before final isolation.

### EXAMPLE 7

Isolation of ddI

The ddI solution is distilled under vacuum at an internal batch temperature of 20-40° C. The distillation is stopped when the concentration of ddI reaches 10-12% w/v based on initial ddA. Typically the pH is 8.1-8.3 at this point. Additional water is added and distillation is then continued until the concentration again reaches 10-12%, and the pH of the ddI slurry is less than 8 (typically 7.8-7.9). The ddI suspension is cooled to 0-5° C. and held for at least 1 hour. The cold slurry is filtered and the cake is washed with 0-5° C. water. The mother liquor and aqueous wash can be retained for recycling in another batch.

The cake is washed with 0-5° C. acetone. The solids are dried under vacuum at 45-50° C. to a constant weight. Yields of ˜82% for the first run and 96-99% for four subsequent runs (>96% overall) are expected with mother liquor recycling. The resulting ddI is >99% pure.

### Comparative Example 1

### Comparison of E. coli ADA Activity and Recombinant Human Immobilized ADA

Microbial ADA from recombinant E. coli was partially purified and isolated as an ammonium sulfate suspension. E. coli fermentation broth (42 U ADA/mL, 2 L) was centrifuged and the cell pellet was collected and washed with 1 L 100 mM phosphate buffer pH 7.5. The cells were again centrifuged and resuspended in 2 L of the above buffer plus 20% glycerol. The cells were passed once through a microfluidizer. Cell debris was removed by centrifugation and the resulting supernatant was concentrated by ultrafiltration through a 30,000 MWCO cassette. The enzyme was concentrated to a final titer of 390 U/ml. Ammonium sulfate was added to a 50% saturation, the resulting precipitate was collected by centrifugation and re-suspended in 100 ml deionized water. The final titer of the slurry was 740 U/ml; the protein content of the slurry was ˜75 mg/ml.

The following Table describes the conversion of a 52-60 g/L solution of ddA in water to ddI using the microbial adenosine deaminase as described above versus an example using the recombinant human ADA immobilized enzyme following the general procedure in Example 4. As can be seen from the results of Table 2, the recombinant human immobilized enzyme is significantly more stable than microbial enzymes and allows for the completion of the reaction with less units of enzyme and in a short period of time.

TABLE 2
Comparative Yields of ddA from E. coli Versus
Recombinant Human ADA

| | Enzymatic | Reaction | Reaction | Conversion of |
|---|---|---|---|---|
| Source of | Activity | Temperature | Time | ddA to ddI |
| ADA | (U/g ddA) | (° C.) | (hours) | (%) |
| E. coli | 87.5 | 40 | 16 | 16 |
| (NH₄)₂SO₄ | | | | |
| suspension | | | | |
| E. coli | 125 | 37 | 24 | 26 |
| (NH₄)₂SO₄ | | | | |
| suspension | | | | |
| Recombinant | 40 | 30 | 4.5 | 100 |
| human | | | | |
| immobilized | | | | |
| ADA | | | | |
| Recombinant | 40 | 40 | 1.5 | 100 |
| human | | | | |
| immobilized | | | | |
| ADA | | | | |

Thus, while there have been described what are presently believed to be the preferred embodiments of the present invention, other and further embodiments, modifications, and improvements will be known to those skilled in the art, and it is intended to include all such further embodiments, modifications, and improvements and come within the true scope of the claims as set forth below.

What is claimed is:

1. A method of making didanosine (ddI) comprising the steps of:
(a) obtaining an enzyme expressing ddA deaminase activity;
(b) immobilizing the enzyme onto an insoluble support;
(c) contacting the enzyme with a dideoxyadenosine (ddA) solution of at least about 1% weight volume ddA in water for a time and under conditions to produce a ddI solution; and
(d) isolating the ddI from the ddI solution.

2. The method of claim 1, wherein the ddA solution in the contacting step is from about 2% to about 10% weight volume ddA in water.

3. The method of claim 1, wherein a pH during the contacting step is from about 8.0 to about 9.5.

4. The method of claim 3, wherein substantially all of the ddI resists precipitation out of the ddI solution in the contacting step.

5. The method of claim 1, wherein the insoluble support is functionalized to allow attachment of the enzyme thereto.

6. The method of claim 5, wherein the attachment of the enzyme to the insoluble support is achieved using an activating agent.

7. The method according to claim 1, wherein the enzyme is human adenosine deaminase (ADA).

8. The method of claim 7, wherein the ADA has the amino acid sequence of SEQ ID NO: 1, or conservative variations thereof.

9. The method of claim 7, wherein the ADA is coded for by a nucleotide having SEQ ID NO: 2, SEQ ID NO:3, or conservative variations thereof.

10. The method according to claim 1, wherein the obtaining step includes expressing human adenosine deaminase (ADA) or a conservative variant thereof in a transformed organism and isolating the ADA from the organism.

11. The method of claim 10, wherein the transformed organism is E. coli.

12. The method of claim 10, wherein the insoluble support is functionalized to allow attachment of the enzyme thereto.

13. The method of claim 12, wherein attachment of the enzyme to the insoluble support is achieved using an activating agent.

14. The method of claim 10, wherein an activity of the enzyme immobilized on the insoluble support is at least about 40 U/g.

15. The method of claim 10, wherein a pH during the contacting step is from about 7.5 to about 9.5.

16. The method of claim 10, wherein said contacting step is a continuous process performed using a packed column.

17. The method of claim 10, wherein the ddA solution in the contacting step is from about 4% to about 15% weight volume ddA in water.

18. The method of claim 17, wherein the ddA solution is from about 5% to about 8% weight volume ddA in water.

19. The method of claim 10, wherein the isolating step includes sequentially distilling the ddI solution and adding water until a ddI slurry in aqueous mother liquor is obtained and the pH is less than about 8.

20. The method of claim 10, further comprising the steps of:
(a) retaining a reaction mother liquor after the isolating step; and
(b) repeating the contacting step at least once using the reaction mother liquor to prepare the ddA solution; and
(c) repeating the isolating step at least once.

21. The method of claim 20, wherein the isolating step produces a yield of at least about 96% ddI that is at least about 99% pure.
